(19) United States
(12) Patent Application Publication
(10) Pub. No.: US 2017/0274127 A1
(43) Pub. Date: Sep. 28, 2017

(54) All 4 Babies

(71) Applicants: Da'Nae Mitchell, Kansas City, MO (US); Monique Watkins, Kansas City, MO (US)

(72) Inventors: Da'Nae Mitchell, Kansas City, MO (US); Monique Watkins, Kansas City, MO (US)

(21) Appl. No.: 15/458,585

(22) Filed: Mar. 14, 2017

Related U.S. Application Data
(60) Provisional application No. 62/313,062, filed on Mar. 24, 2016.

(51) Int. Cl. A61M 1/06; F24C 15/26; F24C 7/10; A61J 13/00

(52) U.S. Cl. CPC A61M 1/062; A61J 13/00; F24C 15/26; F24C 7/10; A47J 43/044

(57) ABSTRACT

The portable infant bottle filler and warmer machine may make it much easier to prepare bottles of formula or breast milk when feeding babies. It may be used at home, in a hospital, and while traveling. The machine mixes the appropriate amount of formula and water based on the ounces needed for the baby, and once mixed, warms the liquid to the appropriate temperature for feeding. The machine may also be used to pump and manage breast milk production. Pumped breast milk is kept cool until ready for use, and is then warmed to the necessary temperature. Other items such as wipes may be warmed within the machine as well for the comfort of the baby.

### CROSS-REFERENCES TO RELATED APPLICATIONS (IF ANY)

This application is the non-provisional application claiming the priority date of provisional application 62/313,062 filed Mar. 24, 2016 which is incorporated by reference.

### BACKGROUND OF THE INVENTION

1. Field of the Invention

The present invention relates generally to the field of infant care and more specifically relates to a machine which helps parents with specific tasks such as making and warming bottles to the correct temperature.

2. Description of the Related Art

Caring for a new baby entails a great deal of work, and new parents are often overwhelmed by all that is required once being discharged from the hospital. As every parent knows, it is not easy to fix a baby's bottle, especially in the middle of the night when the baby is crying. It takes time and concentration to get the formula mixed correctly and the temperature of the water just right. New parents especially worry about heating up the bottles in pans of water or using the microwave, as these methods may make the bottles too hot/destroy the bottles, while some parents believe that the microwave process is detrimental for the baby. When babies are breast fed, there are similar concerns when breast milk is transferred to bottles. Babies cry when their diapers need changing, and they may cry louder when a cold diaper wipe is used to clean their bottoms. All the activities involved with caring for a baby become more complicated when the parents want or need to travel with a child under the age of two.

Various attempts have been made to solve the problems which may be found in the related art but have thus far been unsuccessful. A need exists for a reliable portable infant bottle filler and warmer device to avoid the above-mentioned problems.

### BRIEF SUMMARY OF THE INVENTION

In view of the foregoing disadvantages inherent in the known art, the present invention provides a novel portable infant bottle filler and warmer device. The general purpose of the present invention, which will be described subsequently in greater detail, is to provide a portable bottle maker that may be used by mothers who are breastfeeding as well as parents using formula. It may keep the breast milk cool to preserve its freshness, may mix formula bottles accurately, and may warm both breast milk and formula bottles to the perfect temperature for feeding a baby. The portable infant bottle filler and warmer may also include a newborn feeding schedule monitor. The breastfeeding portion of the machine may also monitor the amount of breastmilk produced by the mother. The device may also be used to warm diaper wipes, making babies more comfortable when being changed.

Inside the machine there may be a device to measure and dispense out the correct amount of formula with the selected ounces of the bottle to be made. There may also be a blending tool or instrument (mixing chamber) which mixes the water and formula. Once the formula and water is mixed, it flows through the warming tube (heating system) and is dispensed into the bottle. On the machine there is a small blue button which is the power button. Below the power button are the instructions for use. Formula and water may be stored in the bottle maker and may be replaced when the formula and water is low.

The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, portable infant bottle filler and warmer, constructed and operative according to the teachings of the present invention.

FIG. 1 shows a perspective view illustrating the portable infant bottle filler and warmer indicating a charge port location, a dispensing nozzle for premixed formula, a clear door for viewing inside the machine, a bottle, and a handle for carrying the machine according to an embodiment of the present invention.

FIG. 2 shows a perspective view illustrating the portable infant bottle filler and warmer indicating the charge port location, the dispensing nozzle for premixed formula, the clear door for viewing inside the machine; the bottle in position to be filled by the dispensing nozzle, the handle for carrying the machine, and a tube which fastens to a breast pump cup according to an embodiment of the present invention.

FIG. 3 shows a perspective view illustrating the portable infant bottle filler and warmer indicating a control panel, the tube fastened to the breast pump cup, the handle for carrying the machine, and depicting a portion which is opened where formula and water are stored according to an embodiment of the present invention.

FIG. 4 shows a perspective view illustrating the portable infant bottle filler and warmer indicating the handle for carrying the machine, the bottle behind the clear door for easy viewing bottle content, the tube connecting the system to breast pump cup, and USB charging port useful for powering system according to an embodiment of the present invention.

FIG. 5 shows a perspective view illustrating an in-use touch screen displaying different available mode.

The various embodiments of the present invention will hereinafter be described in conjunction with the appended drawings.

### DETAILED DESCRIPTION

As discussed above, embodiments of the present invention relate to infant care and more particularly to a machine which helps parents with specific tasks such as making and warming bottles to the correct temperature.

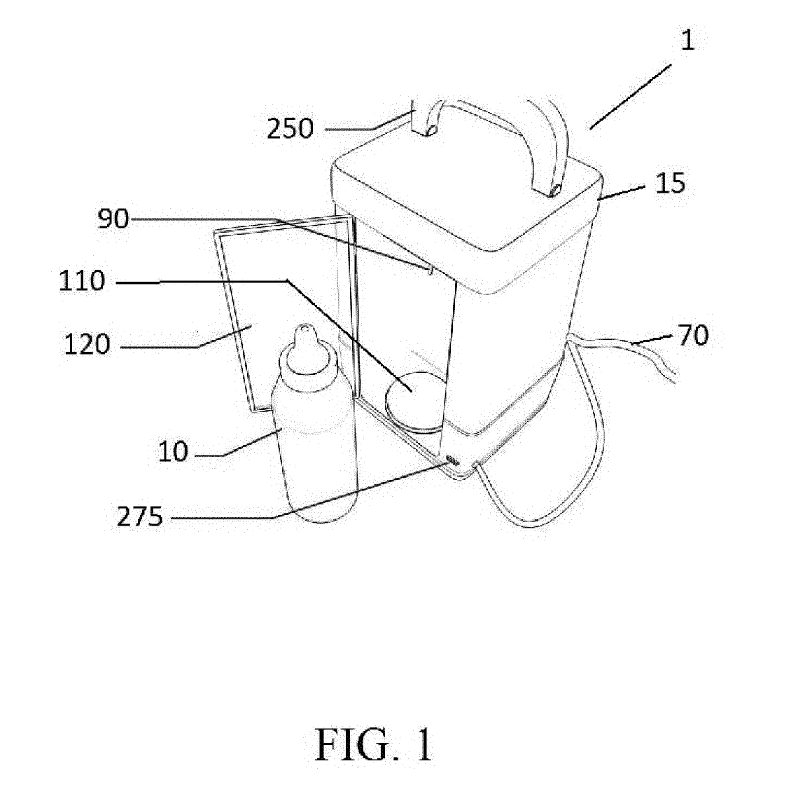

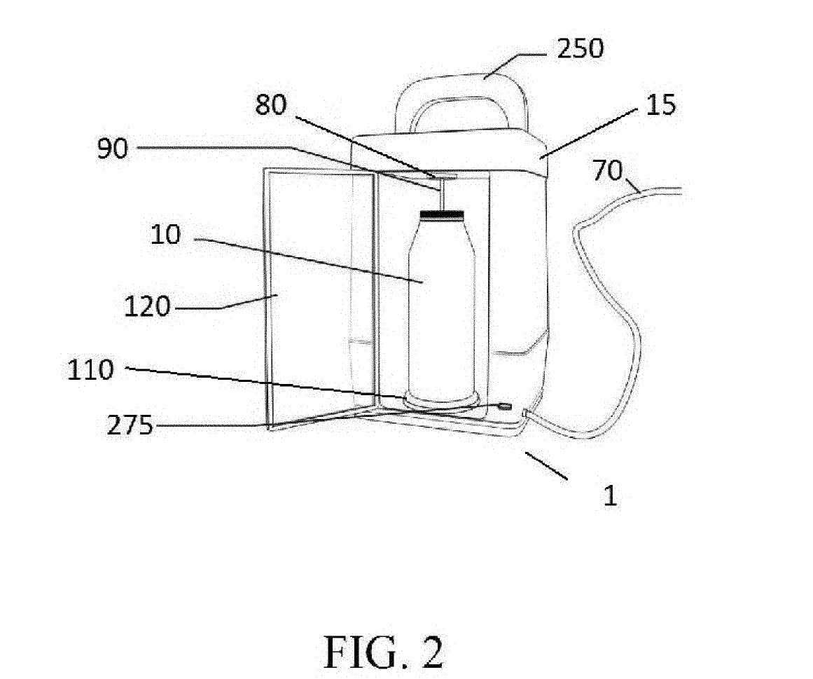

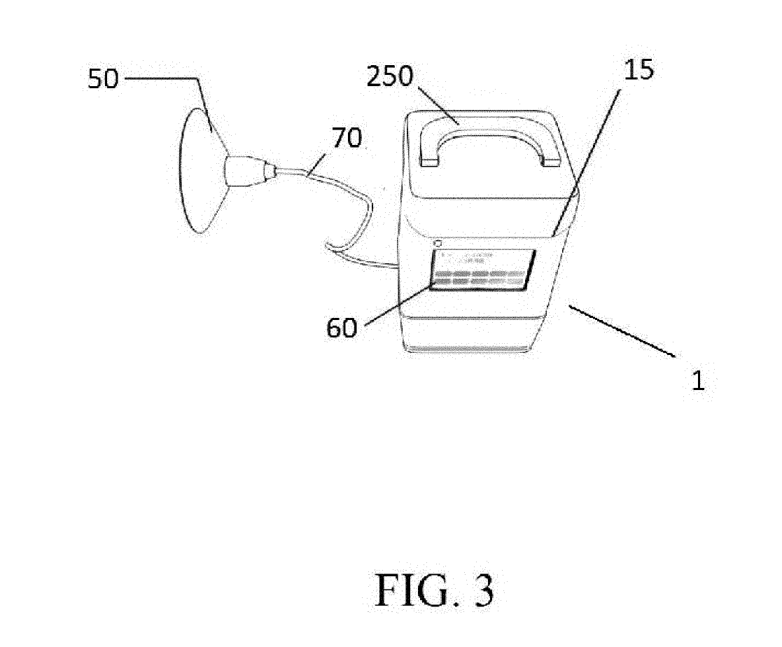

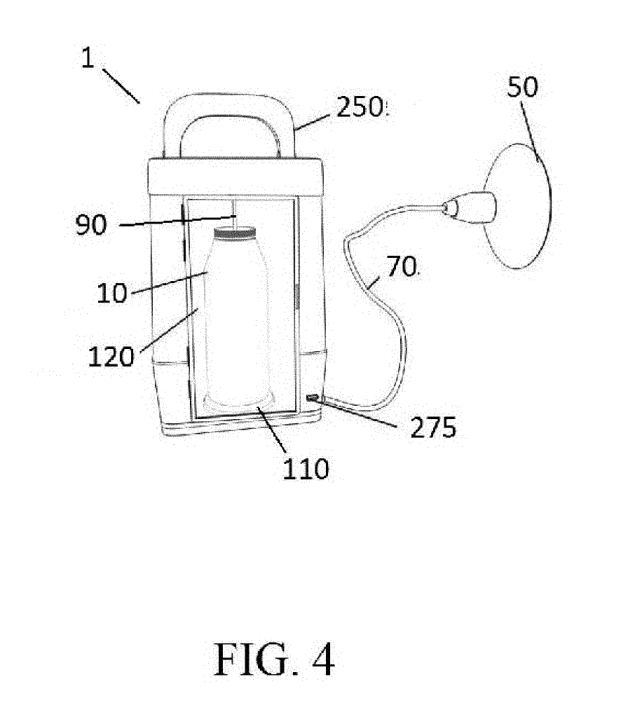

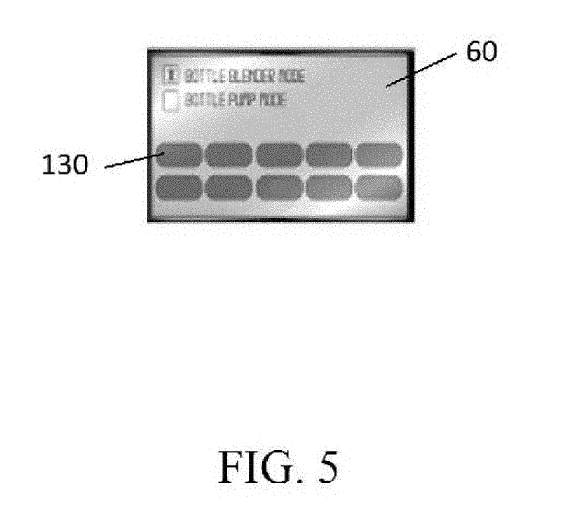

Referring now to the drawings FIGS. 1-5, showing various views of the portable infant bottle filler and warmer system designed to make feeding and changing a baby less complicated for parents when they at home and traveling. This machine may be used as a breast pump. After placing a bottle 10 with a flat top 15 in the machine 1 and placing the breast shield 50 on one breast, the machine 1 is turned on and set to breast pump mode as shown on the touchscreen 60 in FIG. 5. The user then selects a comfortable pressure level, and the pumped milk is transferred from the breast to the bottle through the attached tubing 70, making it easy to monitor the mother's output. Once in the bottle 10, it can be chilled, by selecting the chilling function, until it is needed. Preparing a bottle 10 of formula is just as easy. On the inside of this machine 1, there are separate storage compartments for dry formula and water, and the ingredients may be dispensed in the correct amount needed based on the number of ounces for the bottle 10 being made which is accessible by lifting up a lid 15 in the top of the machine 1. There is a blending tool 80 to mix the water and formula before it is released through the warming tube 90, so it will be at the perfect serving temperature when it flows in the baby's bottle.

On the machine there is a small blue button which is the power button. Below the power button are the instructions for use in the preferred embodiment. Formula and water may be stored in the machine 1 and may be replaced when the formula and water is low.

The warming system is set to stay at 98.5 Farenheight, and may be used for heating baby food, wipes, and bottles of breast milk. The item to be warmed is placed on a platform 110 inside a door 120, the door 120 is closed, and the warm button 130 is pressed to heat the item within seconds. The door 120 is on hinges and is located on the side of the housing. A case containing a number of baby wipes may be placed on the platform to keep them from drying out and to warm more than one at a time. This machine 1 has a rectangular housing, has a touch screen control panel 60, a carrying handle 250 and a USB port 275 for plugging in the power cord. The handle 250 would be located on the top of the housing. The machine 1 can have a rechargeable battery as a power source.

A car charger is also included, making it convenient to use on the road. There may also be a light inside the door, which comes on automatically, making it easier to see when using the machine in the middle of the night. It may be available in a plurality of colors. The original version may be compact enough to fit easily in a diaper bag, and there may be a larger version, portable infant bottle filler and warmer for parents of multiples and parents with more than one baby close to the same age.

The unique features of this invention will provide the following benefits for all consumers: a convenient and portable way to prepare baby bottles and to serve them as the ideal temperature. Can be used to pump breast milk directly into bottles 10 discreetly. Bottles 10 will be chilled to preserve the freshness of the breast milk and warmed before being given to the baby. All types of bottles 10 can be used with machine, and bottles 10 with flat tops would be reserved for breast milk. Bottles will be prepared without spills and without air bubbles. Will be used for warming baby food, chilled breast milk, and diaper wipes. Features helpful nutritional information and facts about bottle and breastfeeding babies. Can be used to monitor newborn feedings, making it perfect for hospital usage, as well as the output of a mother's breast milk. The device is easy to use and to clean.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claim:

1. A portable infant bottle filler and warmer device comprising:
A housing with platform, a control panel, a warming tube, with a door.

2. A device according to claim 1 further comprising:
Having a tube connected to a breast pump and breast shield.

3. A device according to claim 1 further comprising:
Having the pump pressure controlled by the control panel.

4. A device according to claim 1 further comprising:
Having breast milk pumped for a breast flowing through the tube through the heating tube to a bottle placed on the platform.

5. A device according to claim 1 further comprising:
Having a compartment for water and formula.

6. A device according to claim 5 further comprising:
Having the housing have a lid and having the compartment be the lid.

7. A device according to claim 5 further comprising:
Having a blending tool mix the formula and the water.

8. A device according to claim 7 further comprising:
Having the mixed formula pumped into a bottle through the heating tube.

9. A device according to claim 1 further comprising:
Having a USB power to provide power.

10. A portable infant bottle filler and warmer device comprising:
A housing with platform, a lid, a control panel, a warming tube, with a door, having a tube connected to a breast pump and breast shield with the pump pressure controlled by the control panel, having a compartment for water and formula in the lid, and having a blending tool mix the formula and the water.

11. A device according to claim 10 further comprising:
Having breast milk pumped for a breast flowing through the tube through the heating tube to a bottle placed on the platform.

12. A device according to claim 10 further comprising:
Having the mixed formula pumped into a bottle through the heating tube.

13. A device according to claim 10 further comprising:
Having a USB power to provide power.
